(19) United States
(12) Patent Application Publication
(10) Pub. No.: US 2011/0207627 A1
(43) Pub. Date: Aug. 25, 2011

(54) EX VIVO THERAPEUTICS SCREENING OF LIVING BONE MARROW CELLS FOR MULTIPLE MYELOMA

(75) Inventors: Lance A. Liotta, Bethesda, MD (US); Emanuel F. Petricoin, III, Gainesville, VA (US); Virginia Espina, Rockville, MD (US)

(73) Assignee: GEORGE MASON UNIVERSITY

(21) Appl. No.: 13/057,978

(22) PCT Filed: Aug. 12, 2009
(86) PCT No.: PCT/US09/04608

§ 371 Date: May 9, 2011

Related U.S. Application Data
(60) Provisional application No. 61/088,392, filed on Aug. 13, 2008.
(60) Provisional application No. 61/090,006, filed on Aug. 19, 2008.

(51) Int. Cl. C12Q 1/02; G01N 33/566; C40B 30/04; C12Q 1/37; C12Q 1/48; H01J 49/26

(52) U.S. Cl. 506/9; 435/29; 435/7.92; 435/23; 435/15; 435/7.1; 250/282

(57) ABSTRACT

Methods of selecting a treatment for a patient with multiple myeloma are provided. Prior to commencing a treatment regime, bone marrow aspirates are isolated from a patient and incubated with one or more candidate therapeutics. The methods identify the therapy or combination of therapies most likely to yield the best results for a particular individual. In addition to improving clinical outcome, such theranostic evaluations dramatically reduce health care costs, by avoiding ineffective therapies. Screening assays for identifying treatments for multiple myeloma also are provided.

CROSS-REFERENCE TO RELATED PATENT APPLICATIONS

This application claims the priority benefit of U.S. Provisional Application No. 61/088,392 filed on Aug. 13, 2008 and U.S. Provisional Application No. 61/090,006 filed on Aug. 19, 2008, both of which are hereby incorporated by reference.

### BACKGROUND

Currently, therapeutics are chosen based on population-based clinical trials using broad phenotypic analysis. Targeted approaches attempt to group patients based on larger histologic context (e.g. HER2+ breast cancers). In view of the growing recognition of the individuality of diseases such as cancer, where each patient appears to possess a unique constellation of molecular derangements in their diseased cells and unique constitutional properties of their non- diseased cells, a new opportunity exists to develop approaches and methods whereby each patient acts as his or her own “clinical trial”. Under this rubric, the molecular profile of an individual patient is used to guide both therapy and dosing. Indeed, even combinations of different therapeutics can be selected rationally by methods and workflows that use patient-specific analysis.

Even when tailored for an individual's diseased cells, a therapy may be equally toxic, or more toxic, to non-diseased cells. Furthermore, drug screening using cell culture or animal models may have little relevance to the cellular microenvironment of the living patient. Nor is it practical to test in the same patient multiple drugs or drug combinations in vivo. Thus, there is a substantial need to individualize therapeutic screening for diseased cells in parallel with non-diseased cells in the same patient using an ex vivo assay.

### SUMMARY

In one aspect, a method of selecting a treatment for a patient with multiple myeloma comprises incubating a bone marrow aspirate from said patient with one or more candidate therapeutics; stopping the incubation and applying a fixative to the stopped incubation; isolating CD138(+)(plamacytoid) cells present in said stopped incubation so as to form at least one sample containing CD138(+) cells and at least one other sample containing CD138(−) cells; analyzing the samples for apoptosis induction of cells or phospho-protein signal pathway activation or suppression; and (E) using the analysis to select a treatment that advantageously impacts CD138(+) cells as compared to CD138(−) cells. In one embodiment, the analysis involves considering the efficacy of candidate therapeutics in selectively inhibiting CD138(+) cells. In one example, efficacy can be assessed by measuring the efficiency, specificity or toxicity of candidate therapeutics upon CD138(+) cells as compared to CD138(−) cells.

In one embodiment, the analysis step comprises evaluating the phosphorylation state of two or more endpoints in a signal pathway comprising the target of the candidate therapeutic. In another, the analysis step comprises evaluating the phosphorylation state of two or more endpoints in a signal pathway influencing cell survival, cell death or cell growth. In another, the analysis step comprises assays evaluating caspase cleavage, poly(ADP-ribose) polymerase (PARP) cleavage or dye exclusion/uptake.

In another embodiment, the method further comprises confirming the selection by molecular analysis of a putative target of the selected therapeutic. In one example, the molecular analysis is selected from the group consisting of reverse phase microarray, suspension bead array, ELISA, flow cytometry, immunoasay and high resolution mass spectroscopy. In one embodiment, the isolation step involves sorting CD138(+) cells via FACS or magnetic bead separation. In one example, the isolation step involves sorting CD138(+) cells via magnetic bead separation using a rare earth magnet. In another example, the magnetic bead separation uses a neodymium magnet. It should be recognized that CD138 is just one example of a cell surface antigen that can be used to separate diseased target cells from non-diseased host cells in the bone marrow, after the therapeutic incubation.

In one embodiment, the method further comprises, prior to the incubation step, evaluating the molecular signal pathway of apoptosis, cell survival, autophagy, differentiation, growth factor signaling, cell cycle and cell motility.

In another aspect, methods are provided for identifying potential treatments for multiple myeloma, such methods comprising (A) incubating a bone marrow aspirate from a multiple myeloma patient with one or more candidate therapeutic; (B) stopping the incubation and applying a fixative to the stopped incubation; (C) isolating CD 138(+) cells present in said stopped incubation so as to form at least one sample containing CD138(+) cells and at least one other sample containing CD138(−) cells; (D) analyzing the samples for apoptosis induction of cells or phospho-protein signal pathway activation or suppression; and (E) using the analysis to select a treatment that advantageously impacts CD138(+) cells as compared to CD138(−) cells.

Other objects, features and advantages will become apparent from the following detailed description. The detailed description and specific examples are given for illustration only since various changes and modifications within the spirit and scope of the invention will become apparent to those skilled in the art from this detailed description. Further, the examples demonstrate the principle of the invention and cannot be expected to specifically illustrate the application of this invention to all the examples where it will be obviously useful to those skilled in the prior art.

### BRIEF DESCRIPTION OF THE DRAWINGS

FIG. 1 schematically shows an exemplary workflow for analyzing MM patient samples. A. Bone marrow aspirate and bone marrow core biopsy samples are obtained by standard aspiration technique. B. The aspirate sample is divided into multiple portions and placed in a microtiter plate to which inhibitors, ligands, or combinations of inhibitors and/or ligands are added. The samples are incubated at 37° C. for 4 hours. C. At the end of the incubation period, the sample is stabilized by adding an ethanol-based protein preservative solution. The solution contains kinase and phosphatase inhibitors and reversible cross-linkers which prevent cellular signaling activity. Red blood cells are lysed in a RBC lysis buffer. CD138+ myeloma cells are separated magnetically from the bone marrow milieu. Proteins are extracted from both CD138+ myleoma cells and CD138− non-myleoma cells. D. Reverse phase protein microarrays are constructed from the cellular lysates. Each array is probed with a single primary antibody with subsequent signal amplification and detection. E. Provides a schematic overview of apoptosis which is useful in signal pathway mapping.

FIG. 2 provides an in vitro comparison of viable/non-viable cells post inhibitor treatment. Composite results are provided from 24 different experiments with 3 cell lines and 12 inhibitors. Cells were designated as alive or dead based on trypan blue viability post inhibitor treatment compared to DMSO vehicle alone. Cell viability status was correlated with cleaved forms of apoptotic proteins (Cleaved Caspase 3, 6, 7, 9 or Cleaved PARP) (Wilcoxon non parametric analysis) to identify protein endpoints indicative of cell death. Values on the y-axis represent fold increases in protein activation compared to vehicle control.

FIG. 3 provides ex-vivo treatment response profiles for a female, treatment nave, multiple myeloma patient. Dasatinib caused an increase in NFkB p65 Ser536 for the BMC (bone marrow cells (CD138-)) cell population. Differential effect on death signaling endpoints are noted in Sunitinib treatment. Differential pro survival signaling in IL-6 stimulated cells compared to IGF1R & Rapamycin combination therapy can be seen. Values on the y-axis represent treatment as a percentage of vehicle control. CD138+ cells: triangles; bone marrow cells (BMC): squares.

FIG. 4 provides ex-vivo treatment response profiles for a male multiple myeloma patient treated with thalidomide and dexamethasone. Differential effects on cell population specific to combination treatment selections can be seen. Values on the y-axis represent treatment as a percentage of vehicle control. CD138+ cells: triangles and bone marrow cells; (BMC): squares.

FIG. 5 provides ex-vivo treatment response profiles for a male, treatment naïve, multiple myeloma patient. Patient response to inhibitor and combination treatment is dominated by pro-survival pathway augmentation through phosphorylation of AKT. Values on the y-axis represent treatment as a percentage of vehicle control. CD138+ cells: triangles; bone marrow cells (BMC): squares.

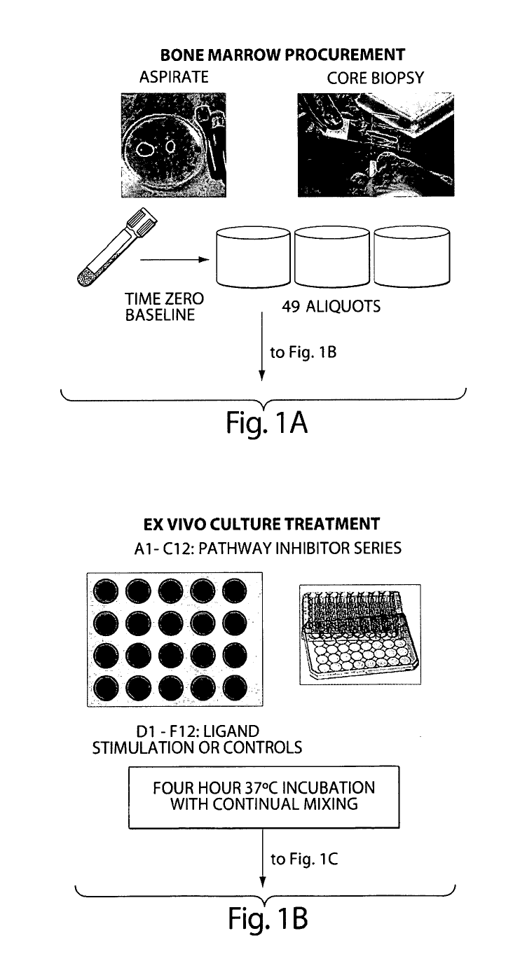

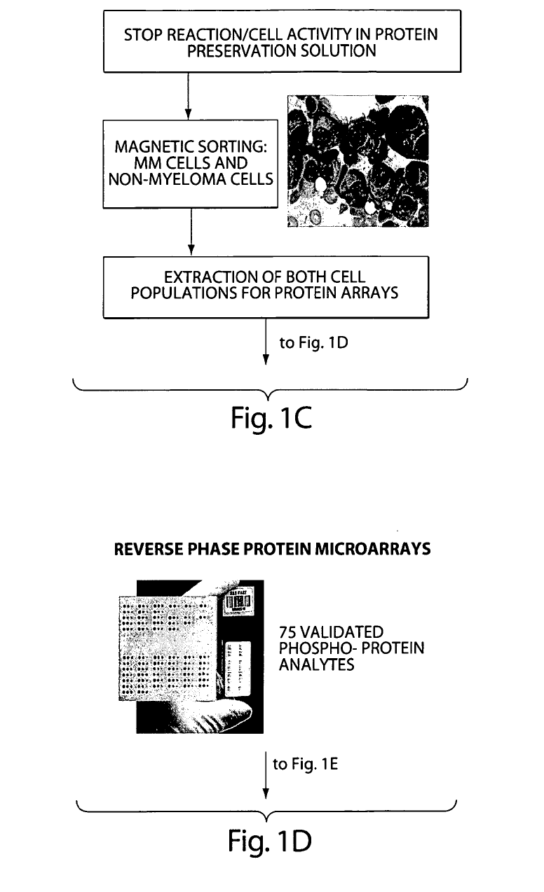

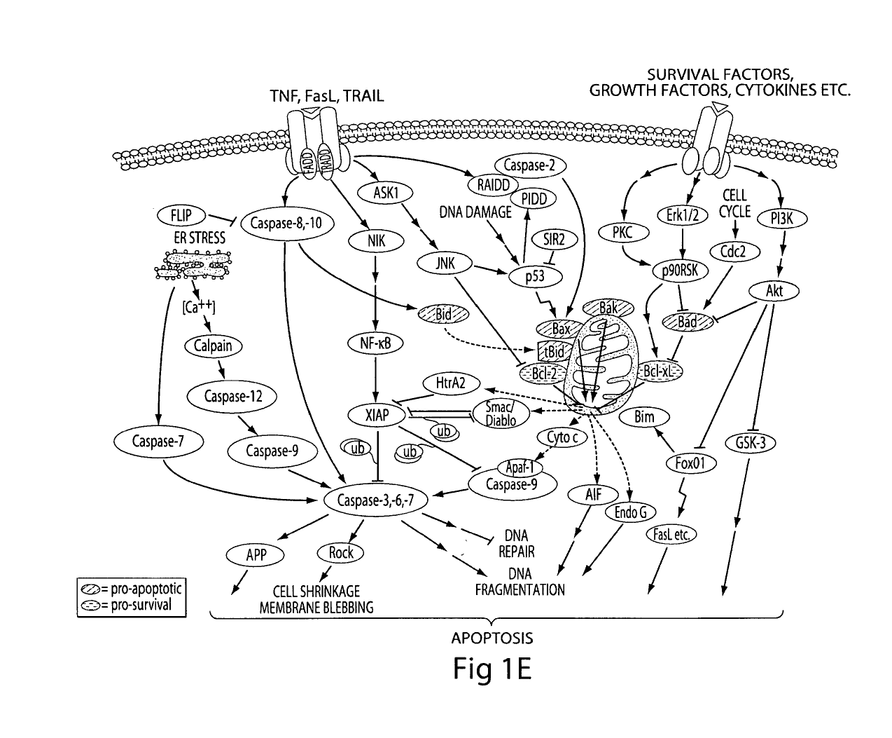

### DETAILED DESCRIPTION

Prior to commencing a treatment regime, patients with multiple myeloma can ascertain which therapy or combination of therapies are most likely to yield the best results for their individual disease. Evaluating the efficacy and side-effects of available treatments prior to in vivo administration maximizes therapeutic potential and minimizes adverse events. In addition to improving clinical outcome, such theranostic evaluations dramatically reduce health care costs by avoiding ineffective therapies.

In one aspect, methods are provided for simultaneously treating both diseased and non-diseased cells of a particular tissue with a multiplicity of drugs and/or drug combinations. Drug efficacy can be measured by evaluating the drug-induced perturbation of the cell signaling network of the treated diseased and normal cells. The methods permit the evaluation of dozens of drugs and a myriad, such as 50 to 100, of protein signal endpoints in living diseased and healthy cells. Importantly, protein molecules of the cells are fixed following ex vivo treatment and prior to cell separation. In so doing, cell separation and isolation can not perturb cellular signaling events and confound results. Thus, the inventive methods enable the rapid elucidation of a candidate therapeutic's, or a therapeutic combination's, capacity to treat multiple myeloma.

In one embodiment, a method of selecting a treatment for a patient with multiple myeloma comprises incubating a bone marrow aspirate from said patient with one or more candidate therapeutics; stopping the incubation and applying a fixative to the stopped incubation; isolating CD138(+) cells present in said stopped incubation so as to form at least one sample containing CD138(+) cells and at least one other sample containing CD138(−) cells; analyzing the samples for apoptosis induction of cells or phospho-protein signal pathway activation or suppression; and (E) using the analysis to select a treatment that advantageously impacts CD138(+) cells as compared to CD138(−) cells. The method provides a means for assessing the efficacy or dosage of a drug on neoplastic and non-neoplastic cells treated in the same physiologic microenvironment. As a result, an optimal therapy for a patient can be selected that kills neoplastic cells while sparing toxicity for non-neoplastic host cells.

### Bone Marrow Aspiration

Bone marrow can be collected via aspiration, which removes a small amount of bone marrow fluid and cells through a needle inserted into a bone. An adequate volume of bone marrow cells can be procured from one standard pelvic bone aspiration, exactly in the manner used for routine hematologic diagnosis. The aspirate containing from 1% to >30% plasmacytoid cells is subdivided, such that each subdivision contains a similar representation of the diseased and non diseased cells.

### Incubation

To identify the most promising therapy, aspirates from an individual's bone marrow can be treated in vitro with a variety of candidate therapeutics. For example, aspirates can be incubated with a panel of molecular targeted inhibitors (e.g. Sunitinib, Dasatinib, Erlotinib), chemotherapeutics (e.g. Dexamethasone, Rapamycin, Bcl-2 inhibitor), exogenous ligands (e.g. SCF, IGF-1 and/or cytokines (e.g. IL-6). Ideally, the candidate therapeutics target a wide range of growth, prosurvival, autophagy and angiogenesis-related pathways. Exemplary candidate therapeutics include, but are not limited to, Avastin (bevacizumab), Gleevec (imatinib), Lapatinib, Iressa, Tarceva, Sutent (Sunitinib), Dasatinib (Sprycel), Nexavar (Sorafenib), Revlimid, Cucurbitacin I, A77 1726, AG 490, AG 1296, AGL 2043, Bcr-abl inhibitor, HNMPA-(AM)3, IGF-1R inhibitor, Lck inhibitor, LFM-A13, TGFβ inhibitor, CD20 antibody, Bortezomib, Carfilzomib, Chloroquine, Dasatinib, Dexamethasone, Erlotinib, Gefitinib, BCL-inhibitor, Honokiol, IGF-1R inhibitor II, Imatinib, Lapatinib, Mek1 & 2 inhibitor, Melatonin, Midostaurin, Nilotinib, NVP-TK1258-CU-2, Nilotinib, Panobinostat, RAD, Rapamycin, Resveratrol, Sorafenib, Sunitinib, IL-6 ligand, IGF-1 ligand and SCF/C-kit ligand. Each of these treatment agents are well known as potential therapeutic agents for cancer.

### Stopping the Incubation

The incubations of bone marrow cells (diseased and non diseased cells together in the same treatment chamber) and candidate therapeutics are stopped prior to isolation of the diseased cells. In one aspect, incubations can be stopped by placing the cells in a preservative that suppresses fluctuations in kinase pathways. Espina et al., Mol Cell Proteomics,7(10):1998-2018 (2008).

### Cell Fixation—Stabilizing Protein Targets for Profiling

To properly elucidate deranged or hyperactive protein signaling networks within a patient's tumor, protein signatures in tissue specimens should be stabilized prior to cell sorting or separation. Since RBC hemolysis, cell separation or centrifugation can perturb the signaling and confound the analysis, cells should be treated with a preservative prior to sorting. Such cell fixation prevents fluctuations in the cellular analytes of interest during cell separation.

In one embodiment, a combination of precipitating fixative, PEG and enzyme inhibitors is used to stabilize protein signatures. Espina et al., Mol Cell Proteomics, 7(10):1998-2018 (2008). This solution effectively a) stabilizes labile signal pathway phosphoproteins, b) preserves cell surface markers for FACS and magnetic sorting, and c) preserves cellular morphology for cytological diagnosis.

### Cell Sorting

CD138 positive cells can be isolated in a variety of ways known in the field. In one example, CD138 positive cells can be isolated using magnetic sorting. See, e.g. Dumont et al. Immunology, 126(4):588-95 (2009); Ng et al., Blood, 108(8):2745-54 (2006). Magnetic activated cell sorting arranges cell populations depending on their surface antigens (CD molecules). The mixture of cells to be separated is incubated with magnetic beads coated with antibodies against a particular surface antigen (e.g., CD138). Cells expressing CD138 thus bind to the magnetic beads. Afterwards, the cell solution is transferred in a column placed in a strong magnetic field. In this step, cells attached to the beads (expressing the CD138) remain on the column, while other cells (not expressing the CD138) flow through. Accordingly, cells can be separated so as to form at least one sample containing CD138(+) cells and at least one other sample containing CD138(−) cells. In one embodiment, rare earth metals, such as cerium, praseodymium, neodymium and samarium, are used as magnets. In a preferred embodiment, a neodymium magnet is used.

In another embodiment, CD138 positive cells can be isolated by fluorescence-activated cell sorting (FACS). See, e.g. Rawstron et al, Haematologica, 93(3):431-8 (2008). Here, bone marrow aspirate cells are fluorescently labeled for CD138. The cell suspension is entrained in the center of a narrow, rapidly flowing stream of liquid. A vibrating mechanism causes the stream of cells to break into individual droplets. Just before the stream breaks into droplets, the flow passes through a fluorescence measuring station where the fluorescent character of interest of each cell is measured. An electrical charging ring is placed just at the point where the stream breaks into droplets. A charge is placed on the ring based on the immediately-prior fluorescence intensity measurement, and the opposite charge is trapped on the droplet as it breaks from the stream. The charged droplets then fall through an electrostatic deflection system that diverts droplets into containers based upon their charge. The stream is then returned to neutral after the droplet breaks off.

### Cell Viability and Proteomic Analysis

Cell viability can be measured in a variety of ways. In one embodiment, a trypan blue assay can be performed at, for example, 0 min, 4 hours, 24 hours, and 48 hours post treatment. Trypan blue staining is commonly used to distinguish dead and live mammalian cells. At each time point cells typically are washed twice with Dulbecco's phosphate buffered saline and then lysed in a 2.5% solution of β-mercaptoethanol in T-PER (Pierce)/2×SDS Tris/Glycine/SDS buffer (Invitrogen).

Cells can be analyzed for apoptosis induction using a variety of techniques known in the field. In some aspects, analyses can comprise measurement of molecular alterations in a pathway that leads to apoptosis or a pathway that suppresses apoptosis In another aspect, proteins can be examined in a western analysis using antibodies specific for epitopes containing specific phosphorylation states. In this regard, reverse-phase protein arrays (RPPA) can be employed. See, e.g. Petricoin et al., Cancer Res. 67(7):3431-40 (2007); Gulmann et al., Clin Cancer Res. 11(16):5847-55 (2005); Paweletz et al., Oncogene. 20(16):1981-9 (2001); Gulmann et al., J Pathol., 218(4):514-9 (2009).

In contrast to the antibody array, reverse-phase protein microarrays do not require labeling of cellular protein lysates and constitute a sensitive high throughput platform for marker screening, pathophysiology investigation and therapeutic monitoring. Briefly, cell lysate is immobilized in an array configuration via a pin-based mircroarrayer onto glass-backed nitrocellulose slides. These applications result in 350-500 μm wide spots each containing the whole cellular protein repertoire corresponding to a given pathological state. Each slide then can be probed with a diagnostic antibody. The name ‘reverse phase’ is derived from the fact that this type of protein microarray immobilizes the protein to be analyzed. This is in contrast to conventional protein arrays that immobilize the antibody probe. RPPA denatures the protein lysate prior to immobilization and thus does not require labeling of the protein to be analyzed. Thus, RPPA measures the relative expression levels of a protein in many samples simultaneously. See Liotta et al., Cancer Cell, 3(4):317-325 (2003), which is hereby incorporated by reference.

In one example, proteomic analysis can be achieved by printing protein lysates onto RPPAs with an Aushon 2470 arrayer and subsequently performing immunoassays with validated antibodies on a Dako autostainer using IRDye 680 (Licor) for fluorescent detection. Exemplary validated antibodies are provided in Table 1. Spot analysis can be performed using Microvigene version 2.9.9.7 (Vigene Tech). Local spot background can be calculated o for each spot, and negative control spot intensities (secondary antibody only) can be measured. Each antibody background corrected spot intensity value can be normalized to total protein.

TABLE 1

| | |
|---|---|
| Antibody | MW (kDa) |

Antibodies to Phospho-Proteins

| | |
|---|---|
| phospho-Tyrosine (P-Y-100) | NA |
| 4E-BP1 (S65) | 15-20 |
| 4E-BP1 (T37/46) | 15-20 |
| 4E-BP1 (T70) | 15-20 |
| 4G10 (anti Phosphotyrosine) | many |
| c-Abl (T735) | 120 |
| c-Abl (Y245) | 135 |
| Acetyl-CoA Carboxylase (S79) | 280 |
| Ack1 (Y284) | 135 |
| Ack1 (Y857/858) | 135 |
| Adducin (S662) | 80, 120, 110 |
| Akt (S473) | 60 |
| Akt (S473) | 60 |
| Akt1/PKB alpha (S473) (SK703) | 60 |
| Akt (T308) | 60 |
| ALK (Y1586) | 80, 220 |
| AMPKalpha1 (S485) | 62 |
| AMPKBeta1 (S108) | 38 |
| Arrestin1 (Beta) (S412) (6-24) | 50 |
| ASK1 (S83) | 155 |
| ATF-2 (T71) | 70 |
| ATF-2 (T69/71) | 70 |
| ATP-Citrate Lyase (S454) | 125 |
| Aurora A (T288)/B (T232)/C (T198) (D13A11) | 35, 40, 48 |
| Bad (S112) | 23 |
| Bad (S136) | |
| Bad (S155) | |
| Bcl-2 (S70) - no longer available | 28 |
| Bcl-2 (S70) (5H2) | 28 |
| Bcl-2 (T56) | 28 |
| Bcr (Y177) | 160, 210 |
| Caspase-3, cleaved (D175) | 17, 19 |
| Caspase-3, cleaved (D175) (5A1) | 17, 19 |
| Caspase-6, cleaved (D162) | 18 |
| Caspase-7, cleaved (D198) | 20 |
| Caspase-9, cleaved (D315) | 35 |
| Caspase-9, cleaved (D330) | 17, 37 |
| Catenin (beta) (S33/37/T41) | 85 |
| Catenin (beta) (T41/S45) | 85 |
| Chk1 (S345) | 56 |
| Chk2 (S33/35) | 62 |
| Cofilin (S3) (77G2) | 19 |
| CREB (S133) | 43 |
| CREB (S133) (1B6) | 43 |
| CrkII (Y221) | 42 |
| CrkL (Y207) | 39 |
| EGFR (S1046/1047) | 175 |
| EGFR (Y845) | 175 |
| EGFR (Y992) | 175 |
| EGFR (Y1045) | 175 |
| EGFR (Y1068) | 175 |
| EGFR (Y1068) (1H12) | 175 |
| EGFR (Y1148) | 175 |
| EGFR (Y1148) | 185 |
| EGFR (Y1173) | 175 |
| EGFR (Y1173) (9H2) | 175 |
| EGFR (Y1173) (53A3) | 175 |
| eIF4E (S209) | 25 |
| eIF4G (S1108) | 200 |
| Elk-1 (S383) | 62 |
| eNOS (S113) | 140 |
| eNOS (S1177) | 140 |
| eNOS/NOS III (S116) | 132 |
| ErbB2/HER2 (Y1248) | 185 |
| ErbB2/HER2 (Y1248) | 185 |
| ErbB3/HER3 (Y1289) (21D3) | 185 |
| ERK 1/2 (T202/Y204) | 42, 44 |
| Estrogen Receptor alpha (S118) | 66 |
| Estrogen Receptor alpha (S118) (16J4) | 66 |
| Etk (Y40) | 76 |
| Ezrin (Y353) | 80 |
| Ezrin (T567)/Radixin (T564)/Moesin (T558) | 75, 80 |
| FADD (S194) | 28 |
| FAK (Y397) (18) | 125 |
| FAK (Y576/577) | 125 |
| FKHR (S256) | 82 |
| FKHRL1 (S253) | 100 |
| FKHR(T24)/FKHRL1 (T32) | 68, 97 |
| alpha-Fodrin, cleaved (D1185) | 150 |
| FRS2-alpha (Y436) | 80-85 |
| Gab1 (Y627) | 110 |
| GSK-3alpha (S21) (46H12) | 51 |
| GSK-3alpha/beta (S21/9) | 46, 51 |
| GSK-3alpha (Y279)/beta (Y216) | 47, 51 |
| GSK-3beta (S9) | 46 |
| Histone H3 (S10) Mitosis Marker | 17 |
| Histone H3 (S28) | 17 |
| IGF-1 Rec (Y1131)/Insulin Rec (Y1146) | 90 |
| IGF-1R (Y1135/36)/IR (Y1150/51) (19H7) | 90 |
| IkappaB-alpha (S32) | 41 |
| IkappaB-alpha (S32/36) (5A5) | 40 |
| IkappaB-alpha (S32/36) (39A1431) | 42 |
| IRS-1 (S612) | 180 |
| Jak1 (Y1022/1023) | 130 |
| Jak2 (Y1007/1008) | 125 |
| c-Kit (Y703) | 145, 125, 95 |
| c-Kit(Y719) | 120, 145 |
| c-Kit (Y721) | 145, 125, 95 |
| Lamin A, cleaved (D230) | 45, 50 |
| Lck (Y505) | 56 |
| LKB1 (S334) | N/A |
| LKB1 (S428) | N/A |
| MAPK (pTEpY) | 42, 44 |
| MARCKS (S152/156) | 80, 87 |
| MEK1 (S298) | 45 |
| MEK1/2 (S217/221) | 45 |
| Met (Y1234/1235) | 145 |
| MLK3 (T277/S281) | 92, 115 |
| MSK1 (S360) | 90 |
| Mst1 (T183)/Mst2 (T180) | 59 |
| mTOR (S2448) | 289 |
| mTOR (S2481) | 289 |
| NF-kappaB p65 (S536) | 65 |
| NPM (T199) | 38 |
| p27 (T187) | 27 |
| p38 MAP Kinase (T180/Y182) | 40 |
| p40 phox (T154) | 40 |
| p70 S6 Kinase (S371) | 70, 85 |
| p70 S6 Kinase (T389) | 70, 85 |
| p70 S6 Kinase (T412) | 70 |
| p90RSK (S380) | 90 |
| PAK1 (S199/204)/PAK2 (S192/197) | 61-67, 68-74 |
| PAK1 (T423)/PAK2 (T402) | 61-67, 68-74 |
| PARP, cleaved (D214) | 89 |
| Paxillin (Y118) | 68 |
| PDGF Receptor alpha (Y754) (23B2) | 198 |
| PDGF Receptor beta (Y716) | 190 |
| PDGF Receptor beta (Y751) | 190 |
| PDK1 (S241) | 63 |
| PKA C (T197) | 42 |
| PKC alpha (S657) | 82 |
| PKC alpha/beta II (T638/641) | 80, 82 |
| PKC (pan) (betaII S660) | 78, 80, 82, 85 |
| PKC delta (T505) | 78 |
| PKC theta (T538) | 79 |
| PKC zeta/lambda (T410/403) | 76 |
| PKR (T446) | 74 |
| cPLA2 (S505) | 110 |
| PLCgamma1 (Y783) | 155 |
| PLK1 (T210) | 68 |
| PRAS40 (T246) | 40 |
| PRK1 (T774)/PRK2 (T816) | 120, 140 |
| Progesterone Receptor (S190) | 90, 118 |
| PTEN (S380) | 54 |
| Pyk2 (Y402) | 116 |
| A-Raf (S299) | 68 |
| B-Raf (S445) | 95 |
| c-Raf (S338) (56A6) | 74 |
| Ras-GRF1 (S916) | 155 |
| Ret (Y905) | 175 |
| RSK3 (T356/S360) | 90 |
| S6 Ribosomal Protein (S235/236) (2F9) | 32 |
| S6 Ribosomal Protein (S240/244) | 32 |
| SAPK/JNK (T183/Y185) | 46, 54 |
| SEK1/MKK4 (S80) | 44 |
| Shc (Y317) | 46, 52, 67 |
| SHIP1 (Y1020) | 145 |
| SHP2 (Y542) | 70 |
| SHP2 (Y580) | 70 |
| Smad1 (S/S)/Smad5 (S/S)/Smad8 (S/S) | 60 |
| Smad2 (S465/467) | 58 |
| Smad2 (S245/250/255) | 60 |
| Src Family (Y416) | 60 |
| Src (Y527) | 60 |
| Stat1 (Y701) | 84, 91 |
| Stat1 (Y701) | 92 |
| Stat3 (S727) | 79, 86 |
| Stat3 (Y705) (9E12) | 92 |
| Stat3 (Y705) (D3A7) | 79, 86 |
| Stat5 (Y694) | 90 |
| Stat6 (Y641) | 110 |
| Syk (Y525/526) | 72 |
| Tuberin/TSC2 (Y1571) | 200 |
| Tyk2 (Y1054/1055) | 140 |
| Vav3 (Y173) | 95 |
| VEGFR 2 (Y951) | 230 |
| VEGFR 2 (Y996) | 230 |
| VEGFR 2 (Y1175) (19A10) | 230 |
| Zap-70 (Y319)/Syk (Y352) | 70, 72 |

Antibodies to Total Proteins

| | |
|---|---|
| 14-3-3 zeta, gamma, eta | 27 |
| 4E-BP1 | 15-20 |
| Abl SH2 domain | 140, 210 |
| Actin, Beta | 45 |
| Akt | 60 |
| Akt2 (5B5) | 60 |
| Albumin | 67 |
| Aldehyde Dehydrogenase 1 | 55 |
| Aldehyde Dehydrogenase (ALDH) | 55 |
| Aldehyde Dehydrogenase 2 (ALDH2) | 56 |
| Androgen Receptor | 110 |
| Annexin I | 38 |
| Annexin II | 36 |
| ANT (N-19) | 33 |
| Apaf-1 | 130, 140 |
| APC2 Ab-1 | 92 |
| Apolipoprotein D | 24 |
| Atg5 (part of Autophagy Ab Sampler #4445) | 55 |
| Atg12 (part of Autophagy Ab Sampler #4445) | 16, 53 |
| Aurora A/AIK | 48 |
| Axin1 (C76H11) | 110 |
| Bad | 23 |
| Bak | 25 |
| Bax | 20 |
| Bcl-2 | 28 |
| Bcl-xL | 30 |
| Beclin-1 (part of Autophagy Ab Sampler #4445) | 60 |
| Biliverdin Reductase (BVR) | 33/41-42 |
| BLVRB (biliverdin reductase B) (2F4) | 37 |
| Bmi-1 (10C7.2) | ~33 |
| Bub3 | 40 |
| E-Cadherin | 135 |
| N-Cadherin | 140 |
| Calreticulin (FMC 75) | 48 |
| Caspase-3 | 17, 19, 35 |
| Caspase-7 | 20, 35 |
| Caspase-8 (1C12) | 18, 43, 57 |
| Caspase-8 | 54, 55 |
| Caspase-9 | 17, 35, 37, 47 |
| Catenin(beta) | 92 |
| Cathepsin B (G60) | 39-42 |
| CD3 epsilon | 20-25 |
| CD3 zeta (1D4) | 21 |
| CD3 zeta (8D3) | 16, 32 |
| CD24 (FL-80) | 45 |
| CD24 (GPI-linked surface mucin) Ab-2 (SN3b) | 30-70 |
| CD44 (156-3C11) | 80 |
| CD45 (BRA-55) | 180-240 |
| CD45 | 180-220 |
| CD133 (W6B3C1) | 120 |
| CDK2 (78B2) | 33 |
| Cofilin (D59) | 19 |
| Collagen Type I (NFI/20) | 70-90 |
| Complement factor H | 150 |
| Cox-2 - no longer available | 72 |
| Cox-2 (33) | 70 |
| CREB | 43 |
| Cripto | 18, 20 |
| Crystallin, alpha/Beta | 20 |
| Cu/Zn Superoxide Dismutase (SOD) | 19/23 |
| Cyclin A (BF683) | 55 |
| Cyclin B1 (V152) | 60 |
| Cyclin D1 (G124-326) | 36 |
| Cyclin D1 (DCS6) | 36 |
| Cyclin E (HE12) | 50 |
| Cytochrome C | 14 |
| Cytokeratin 8 | 54 |
| DGK | 83 |
| DKK1 | 30, 35 |
| Dvl2 (30D2) | 90-95 |
| Dvl3 | 88-93 |
| EGFR | 175 |
| EGFR (L858R Mut-Spec) | 175 |
| eIF4G | 220 |
| eNOS | 140 |
| ErbB2/HER2 | 185 |
| ErbB2/HER2 (44E7) | 185 |
| c-ErbB2/HER2 | 185 |
| c-ErbB2 (cytoplasmic domain) (N3/D10) | 185 |
| c-ErbB2/HER2 P185 (e2-4001) | 185 |
| ErbB3/HER3 (1B2) | 185 |
| ErbB4/HER4 (111B2) | 180 |
| ERK 1/2 | 42, 44 |
| Estrogen Rec alpha (62A3) | 66 |
| Estrogen Rec alpha (1D5) | 66 |
| FAK | 116 |
| Filaggrin | 40 |
| GFAP | 50 |
| GRB2 | 25 |
| GSK-3beta | 46 |
| HBB (Hemoglobin, beta) | 16 |
| Heme-Oxygenase-1 | 32 |
| Heparanase 1 | 65 |
| HIF-1alpha (54) | 120 |
| Histone Deacetylase 1 (HDAC1) | 62 |
| Histone Deacetylase 3 (HDAC3) | 49 |
| Histone Deacetylase 4 (HDAC4) | 140 |
| Histone H3, Di-Methyl (Lys9) | 15 |
| Histone H3, Di-Methyl (Lys27) | 15 |
| Histone H3, Pan-Methyl (Lys9) | 15 |
| HSP70 (C92F3A-5) | 70 |
| HSP90 (E289) | 90 |
| Ig Light Chain, Kappa | 25 |
| IGF-1 Receptor beta | 98 |
| IGF1 | 20 |
| IGFBP7 | 31 |
| IkappaB-alpha | 41 |
| IL-1beta | 17, 31 |
| IL-2 (YNRhIL2) | 17 |
| IL-6 | 21-28 |
| IL-8 | 11 |
| IL-10 | 21 |
| Insulin Receptor beta (4B8) | 95 |
| IRS-1 | 180 |
| c-Kit (CD117) | 145, 125, 95 |
| LC3B (part of Autophagy Ab Sampler #4445) | 14, 16 |
| Lck | 56 |
| LEDGF (26) | 52/75 |
| Lipocalin-1 (H-45) | 20 |
| LRP6 (C5C7) | 180, 210 |
| MARCKS (2F12) | ≈60 |
| MEK1/2 | 45 |
| MGMT | 21 |
| Microglobulin, beta-2 (FL-119) | 12 |
| MMP-9 | 84, 92 |
| MMP-11 | 55 |
| MMP-14 | 54, 66 |
| Mn Superoxide Dismutase (SOD) | 25 |
| mTOR | 289 |
| Musashi | 35 |
| c-Myc | 57-70 |
| Naked2 (C67C4) | 59, 61 |
| Nanog | 42 |
| NEDD8 | 9 |
| NF-kappaB | 75 |
| Nodal (5C3) | 42 |
| Notch 1 | 130/300 |
| Nucleobindin 1 Precursor | 54 |
| Osteopontin (OPN) | 53 |
| p16 INK4A | 16 |
| Kip1/p27 (57) | 27 |
| p38 MAP Kinase | 40 |
| p53 | 53 |
| p70 S6 Kinase | 70, 85 |
| PAK2 | 61 |
| PDGF Receptor beta | 190 |
| PDGF Receptor Beta (2B3) | 190 |
| PDGF Receptor beta (28E1) | 190 |
| PEDF | 50 |
| PI3-Kinase | 85 |
| PI3-Kinase p110gamma | 110 |
| PKC alpha (M4) | 82 |
| PLC-gamma-1 | 155 |
| PLK1 | 62 |
| PP2A A Subunit | 62 |
| PP2A B Subunit | 62 |
| PTEN | 54 |
| Ras-GRF1 | 155 |
| S100A7 calcium binding protein | 36.85 |
| SAPK/JNK | 46, 54 |
| SDF1Beta | 8.5 |
| Serotonin | 175 |
| SGK1 | 31, 50 |
| Skp1 | 19 |
| Smac/Diablo | 21 |
| c-Src (SRC 2) | 60 |
| Stat3 | 79, 86 |
| Stat5 (3H7) | 90 |
| Stat6 | 110 |
| SUMO-1 | many |
| SUMO-2/3 (18H8) | many |
| Survivin (71G4) | 16 |
| Syndecan-1 (CD138) | 90 |
| Synuclein, a/B (Syn205) | 18 |
| TLR3 | 104 |
| TNF alpha | 17 |
| TNF-R1 (C25C1) | 55 |
| TOPK/PKB | 40 |
| Tubulin, alpha (B-5-1-2) | 50 |
| Tubulin, a/B | 55 |
| UBC3 | 32 |
| Ubiquitin (P4D1) | many |
| VDAC1 (N-18) | 30-35 |
| VEGF Receptor 2 (55B11) | 210, 230 |
| VHL | 24 |
| Vimentin | 57, 50 |
| Wnt5a/B (C27E8) | 45 |
| XIAP Antibody | 53 |
| ZAP-70 (2F3.2) | 70 |

Correlation of Apoptosis Induction with Cell Death.

Based on cell viability, the treated cells can be categorized into two groups, dead and alive. Cells are determined to be alive if less than 35% of the population are dead according to trypan blue assay, whereas they are classified dead if cell death is greater than or equal to 35%. The viability data at 24 and 48 hours are compared with the signal pathway profile at 4 hours. Nonparametric statistical analysis of fold increase in death pathway protein activation compared to vehicle control can be performed on the 2 sample groups.

### EXAMPLES

### Example 1

### Ex Vivo Multiplexed Signal Pathway Inhibitor Treatment of Multiple Myeloma Bone Marrow Aspirates

A workflow using protein kinase signal pathway mapping technology was developed for the ex vivo, short-term drug treatment of fresh, living human multiple myeloma (MM) bone marrow aspirate tumor cells, compared to non MM bone marrow cells for the same patient. The study sought a) to measure the signal pathway perturbations caused by the inhibitor/ligand treatment in individual baseline bone marrow aspirate samples, b) to correlate this with the susceptibility of the ex vivo living sample to apoptosis induction, within 4 hours, by a molecular targeted inhibitor which blocks this pathway, and c) to compare the relative sensitivity of tumor and non tumor bone marrow cells treated in admixture under identical conditions to identify predictive/prognostic protein-based biomarkers.

Human bone marrow aspirates (n=20) and clinical information were collected following an IRB approved protocol from patients providing informed consent. A portion of the aspirate that was not needed for pathological diagnosis was immediately fixed for baseline analysis and the remaining sample was separated into multiple aliquots for ex vivo treatment. Each aliquot was treated for 4 hours with commercial grade inhibitors, FDA approved kinase inhibitors and/or drugs, vehicle control (DMSO) alone or in combination.

Specifically, 50 ul-100 ul bone marrow aspirate was mixed with 200 ul RPMI-1640 serum free media (ATCC) either alone or in combination with specific kinase inhibitors, drugs, or ligands. The inhibitors listed in Table 2 were evaluated. All treatments were performed in duplicate. One aliquot was incubated with RPMI-1640 media only as an untreated control. The treated and untreated bone marrow aspirates were incubated for 4 hours with constant rotation in a 37° C. incubator at ambient humidity and oxygen saturation.

TABLE 2
Inhibitors

17-DMAG
8-hydroxy Guanosine
AKT Inhibitor IV
AKT inhibitor X
AKT inhibitor XI
AMPK Inhibitor, Compound C
BAY 11-7082
Bcr-abl Inhibitor
Bortezomib
Carfilzomib
Caspase-3 Inhibitor VII
Caspase-8 inhibitor I
Caspase-9 inhibitor II
CGP041251 (Midostaurin)
Chloroquine
Cox II Inhibitor
Dasatinib
Dexamethasone
EGFR inhibitor II, BIBX1382
EGFR/Erb-2/Erb-4 Inhibitor
ERK inhibitor II, Negative control
ERK inhibitor III
erlotinib
FGF/VEGF Receptor Tyrosine Kinase Inhibitor, PD173074
Gefitinib
Glycogen Phosphorylase Inhibitor
Granzyme B inhibitor I
HA14-1
HNMPA-(AM)₃ (Insulin Receptor TKI inhibitor)
Honokoil
HSP90 Inhibitor
IGF-1R Inhibitor II
IGF-1R PPP
Imatinib
Imatinib
Jak2 Inhibitor II
Jak3 Inhibitor I
JNK Inhibitor I, (L)-Form
K2529
Lapatinib
LY294002
MAPK Inhibitor PD169316
Mek 1& 2 inhibitor SL327
Melatonin
Melphalan
NVP-BEZ235
NVP-Raf-265
NVP-LBH589
NVP-AMN107 (Nilotinib)
NVP-TKI258-CU-2
PARP Inhibitor XI, DR2313
PD153035 (EGFR Inhibitor)
PD98059 (MEK inhibitor)
PDGF Receptor Tyrosine Kinase Inhibitor I
PI 3-Kα Inhibitor IV
PI 3-Kγ Inhibitor II
Proteasome Inhibitor IX, AM114
RAD001
Rapamycin
Resveratrol
Sorafinib
Src Kinase Inhibitor II
Sunitinib
Terphenyl (FWF416)
VEGF Receptor Tyrosine Kinase Inhibitor III, KRN633
Wortmannin
ZM 336372 (c-Raf inhibitor)

After the 4 hour treatment, a fixative solution (See Espina et al., Mol Cell Proteomics, 7(10):1998-2018 (2008); WO 08/073187, both of which are hereby incorporated by reference) containing phosphatase and kinase inhibitors was added to the cells to stabilize the phosphoproteome and maintain cell morphology. Red blood cells were lysed with a standard RBC hemolysis solution. CD138+ Plasma cells were separated from the bone marrow aspirate microenvironment (non-CD138+ cells) using CD138+ antibody and magnetic beads (Stem Cell Technologies) in a 48 well plate configuration. Neodymium magnets were aligned in a 48 well microtiter plate. The bone marrow aspirates were incubated in a separate 48 well plate positioned directly on top of the microtiter plate containing the neodymium magnets.

Both CD138+ and CD138 negative cell populations were harvested before and after treatment. An aliquot of each cell population was diluted in PBS 1× to prepare a cytospin slide for morphologic analysis. Cytospin preparations were stained with HemaQuik stain.

Reverse phase protein arrays were used to quantitatively map 60 to 75 signal pathway endpoints. The impact of each treatment was measured on the selected endpoint compared to the vehicle control. Comparisons were made for the relative sensitivity between the myeloma cells and the non-myeloma cells for each endpoint and treatment. Induction of cell death was inferred by activation of a series of apoptosis pathway endpoints.

Reverse phase protein microarray construction. A solution of 10% TCEP in T-PER/2× SDS Tris-glycine SDS buffer was used to solubilize the cells and denature the cellular proteins. Reverse phase protein microarrays were printed in duplicate with whole cell protein lysates as described by Petricoin et al., Cancer Res. 67, 3431-40 (2007). Briefly, the lysates were printed on glass backed nitrocellulose array slides (FAST Slides Whatman, Florham Park, N.J.) using an Aushon 2470 arrayer equipped with 350 μm pins (Aushon Biosystems, Billerica, Mass.). Each lysate was printed in a dilution curve representing undiluted and 1:4 dilutions. The slides were stored with desiccant (Drierite, W. A. Hammond, Xenia, Ohio) at −20° C. prior to immunostaining.

Reverse phase protein microarray immunostaining. Immunostaining was performed on an automated slide stainer per manufacturer's instructions (Autostainer CSA kit, Dako, Carpinteria, Calif.). Each slide was incubated with a single primary antibody at room temperature for 30 minutes. Each array was probed with a single polyclonal or monoclonal primary antibody (Table 2). The negative control slide was incubated with antibody diluent. Secondary antibody was goat anti-rabbit IgG H+L (1:5000) (Vector Labs, Burlingame, Calif.) or rabbit anti-mouse IgG (1:10) (Dako). Subsequent protein detection was amplified via horseradish peroxidase mediated biotinyl tyramide with chromogenic detection (Diaminobenzidine) per manufacturer's instructions (Dako). Total protein per microarray spot was determined with Sypro Ruby blot stain (Invitrogen) per manufacturer's directions. Sypro ruby and DAB stained slides are imaged on Alpha Innotech's Nova Ray and UMAX Power Look 1120 flatbed scanner respectively.

Antibody validation and phosphoprotein specificity. Primary antibodies were validated prior to use by immunoblotting with complex cellular lysates such as commercial cell lysates or human tissue lysates. Criteria for antibody validation were a) a single band at the correct molecular weight, or b) if two bands were present, 80% of the signal must have been at the correct molecular weight.

Image analysis and spot quantitation. Each array was scanned, spot intensity analyzed, data normalized to total protein/spot, and a standardized, single data value was generated for each sample on the array (Image Quant v5.2, GE Healthcare, Piscataway, N.J.).

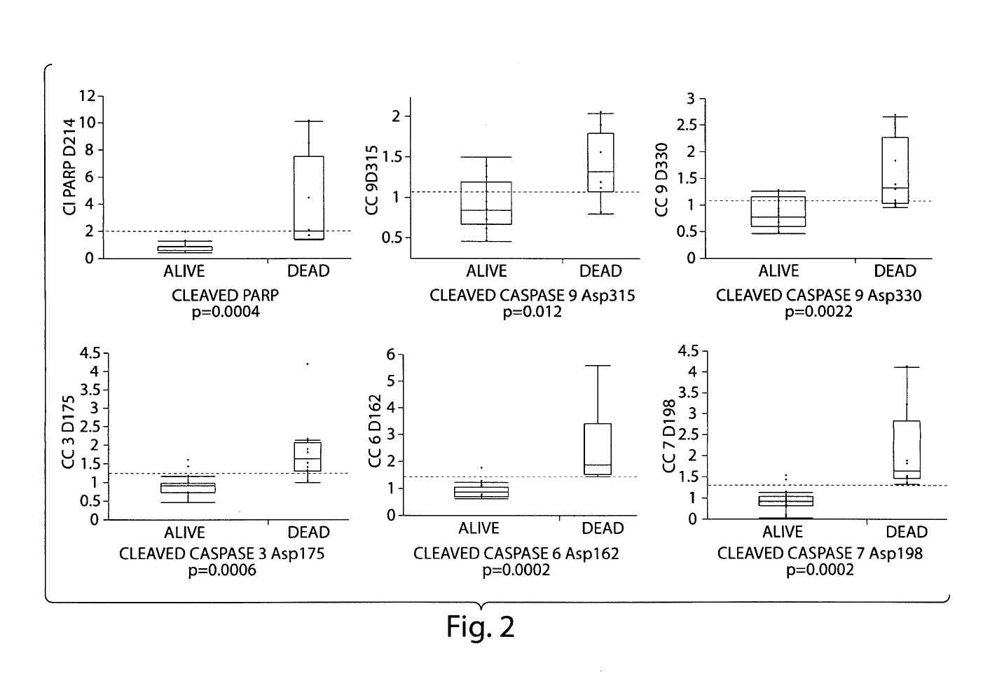

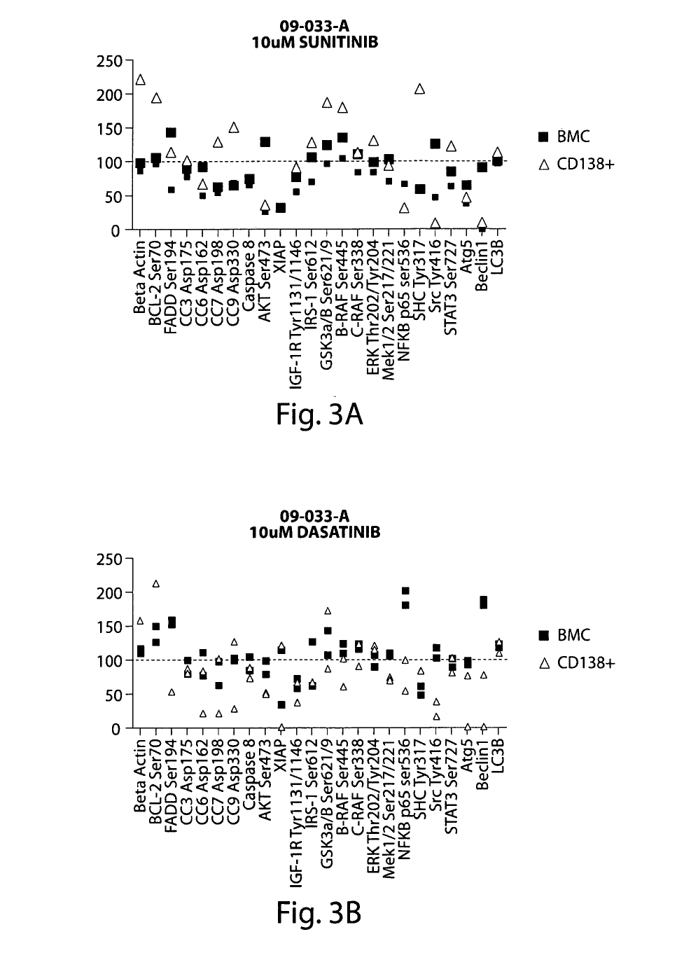

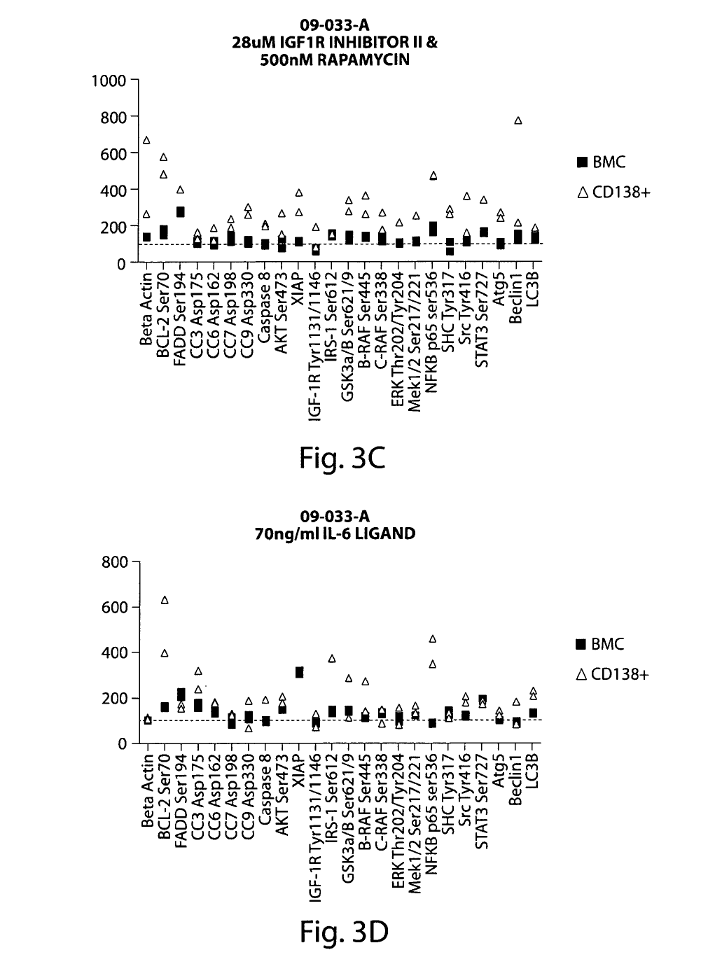

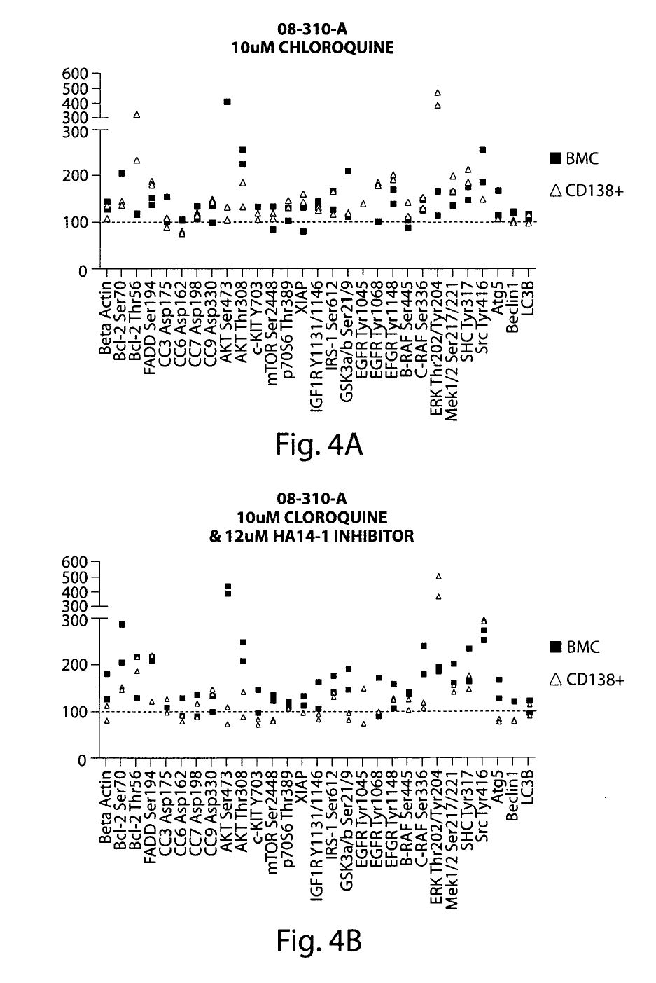

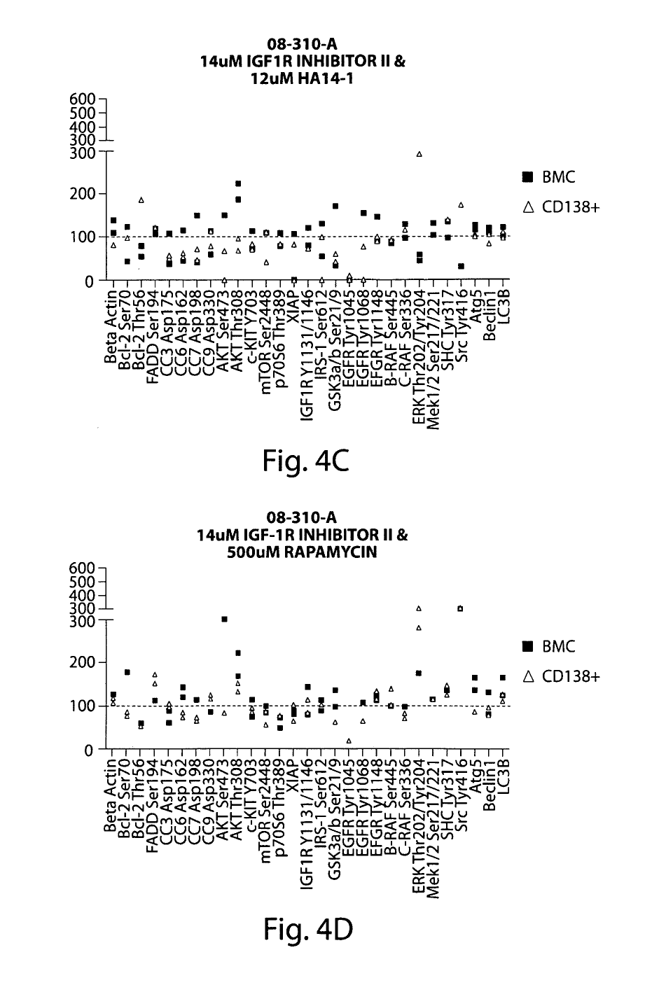

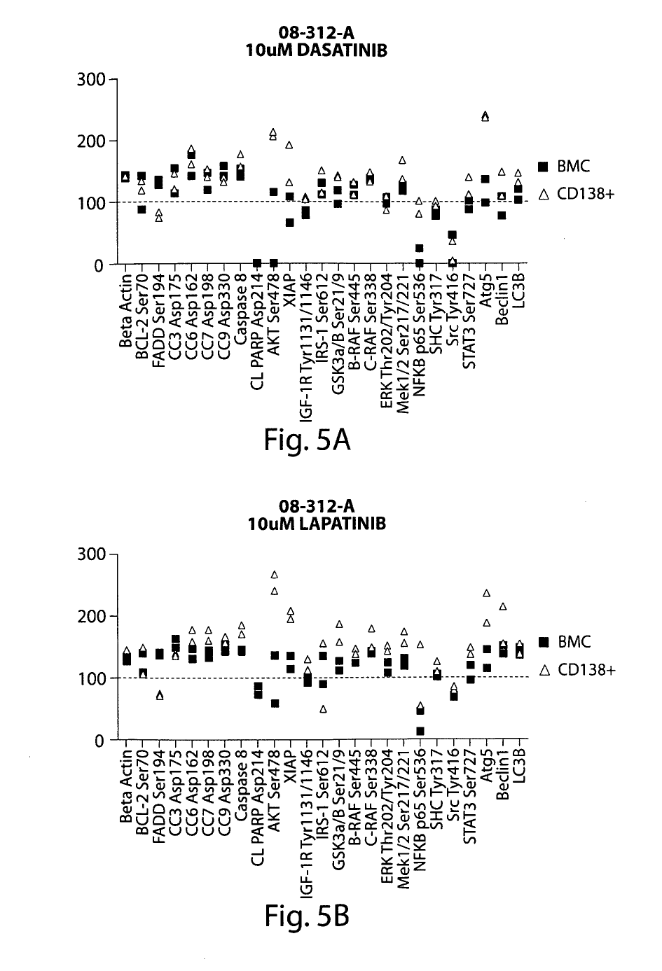

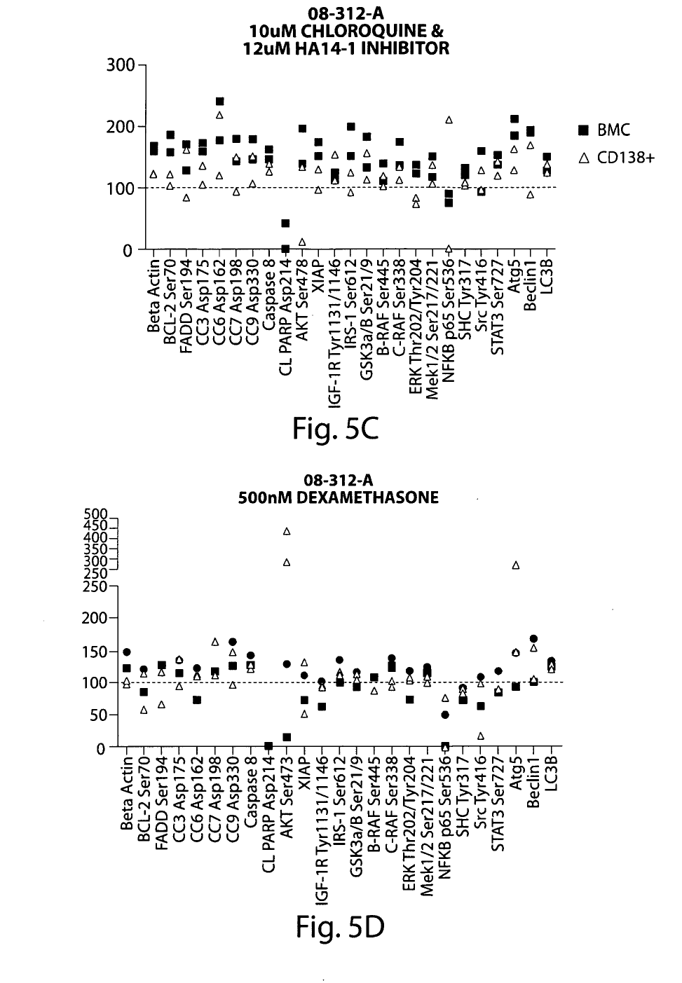

Statistical analysis. The Ward method for two-way hierarchical clustering was performed using JMP v5.0 (SAS Institute, Cary N.C.). Spearman's Rho non-parametric analysis was used to compute the likelihood of correlations between endpoints. When data was normally distributed, two-sample t-test was used (SAS ver9.1.3). Wilcoxon rank sum test was used to compare values between two groups if data was not normally distributed (R ver2.6.1, http://www,R-project.org). p values less than 0.05 were considered significant. The results are shown in FIGS. 2-5.

We claim:

1. A method of selecting a treatment for a patient with multiple myeloma comprising
(A) incubating a bone marrow aspirate from said patient with one or more candidate therapeutics;
(B) stopping the incubation and applying a fixative to the stopped incubation;
(C) isolating CD138(+) cells present in said stopped incubation so as to form at least one sample containing CD138(+) cells and at least one other sample containing CD138(−) cells;
(D) analyzing the samples for apoptosis induction of cells or phospho-protein signal pathway activation or suppression; and
(E) using the analysis to select a treatment that advantageously impacts CD138(+) cells as compared to CD138(−) cells.

2. The method of claim 1, wherein said analysis step comprises evaluating the phosphorylation state of two or more endpoints in a signal pathway comprising the target of the candidate therapeutic.

3. The method of claim 1, wherein said analysis step comprises evaluating the phosphorylation state of two or more endpoints in a signal pathway influencing cell survival, cell death or cell growth

4. The method of claim 1, wherein said analysis step comprises assays evaluating caspase cleavage, poly(ADP-ribose) polymerase (PARP) cleavage or dye exclusion/uptake.

5. The method of claim 1, further comprising confirming said selection by molecular analysis of a putative target of the selected therapeutic.

6. The method of claim 5, wherein said molecular analysis is selected from the group consisting of reverse phase microarray, suspension bead array, ELISA, flow cytometry, immunoasay and high resolution mass spectroscopy.

7. The method of claim 1, wherein said isolation step involves sorting CD138(+) cells via FACS or magnetic bead separation.

8. The method of claim 1, wherein said isolation step involves sorting CD138(+) cells via magnetic bead separation using a rare earth magnet.

9. The method of claim 1, wherein said isolation step involves sorting CD138(+) cells via magnetic bead separation using a neodymium magnet.

10. The method of claim 1, further comprising, prior to the incubation step, evaluating the phosphorylated or activated or post-translationally modified state of signal pathway proteins, receptors or transcription factor proteins.

11. A method of identifying potential treatments for multiple myeloma comprising
(A) incubating a bone marrow aspirate from a multiple myeloma patient with one or more candidate therapeutics;
(B) stopping the incubation and applying a fixative to the stopped incubation;
(C) isolating CD138(+) cells present in said stopped incubation so as to form at least one sample containing CD138(+) cells and at least one other sample containing CD138(−) cells;
(D) analyzing the samples for apoptosis induction of cells or phospho-protein signal pathway activation or suppression; and
(E) using the analysis to select a treatment that advantageously impacts CD138(+) cells as compared to CD138(−) cells.

12. The method of claim 11, wherein said analysis step comprises evaluating the phosphorylation state of two or more endpoints in a signal pathway comprising the target of the candidate therapeutic.

13. The method of claim 11, wherein said analysis step comprises evaluating the phosphorylation state of two or more endpoints in a signal pathway influencing cell survival, cell death or cell growth

14. The method of claim 11, wherein said analysis step comprises assays evaluating caspase cleavage, poly(ADP-ribose) polymerase (PARP) cleavage or dye exclusion/uptake.

15. The method of claim 11, further comprising confirming said selection by molecular analysis of a putative target of the selected therapeutic.

16. The method of claim 15, wherein said molecular analysis is selected from the group consisting of reverse phase microarray, suspension bead array, ELISA, flow cytometry, immunoasay and high resolution mass spectroscopy.

17. The method of claim 11, wherein said isolation step involves sorting CD138(+) cells via FACS or magnetic bead separation.

18. The method of claim 11, wherein said isolation step involves sorting CD138(+) cells via magnetic bead separation using a rare earth magnet.

19. The method of claim 11, wherein said isolation step involves sorting CD138(+) cells via magnetic bead separation using a neodymium magnet.

20. The method of claim 11, further comprising, prior to the incubation step, evaluating the phosphorylated or activated or post-translationally modified state of signal pathway proteins, receptors or transcription factor proteins.
